(19) United States
(12) Patent Application Publication
(10) Pub. No.: US 2015/0050358 A1
(43) Pub. Date: Feb. 19, 2015

(54) IMPLANT SUPPORT COMPOSITION AND METHODS OF USE

(71) Applicant: REGENTS OF THE UNIVERSITY OF MINNESOTA, Minneapolis, MN (US)

(72) Inventors: Wook-Jin Seong, Roseville, MN (US); James Edward Hinrichs, Arden Hills, MN (US); Robert Joseph Schumacher, Edina, MN (US)

(21) Appl. No.: 14/460,024

(22) Filed: Aug. 14, 2014

Related U.S. Application Data
(60) Provisional application No. 61/866,399, filed on Aug. 15, 2013.

(51) Int. Cl. A61L 27/36; A61L 27/58; A61L 27/54; A61L 27/02; A61L 27/22

(52) U.S. Cl. CPC A61L 27/3608; A61L 27/025; A61L 27/227; A61L 27/54; A61L 27/58; A61L 2300/404; A61L 2300/406; A61L 2300/414
USPC 424/549

(57) ABSTRACT

This disclosure describes an implant support composition and methods of using the implant support composition. Generally, the implant support composition includes a filler, an osteoconductive material, an osteoinductive material, and an antimicrobial agent.

### CROSS-REFERENCE TO RELATED APPLICATION

This application claims priority to U.S. Provisional Patent Application Ser. No. 61/866,399, filed Aug. 15, 2013, which is incorporated herein by reference.

### SUMMARY

This disclosure describes, in one aspect, an implant support composition. Generally, the implant support composition includes a filler, an osteoconductive material, an osteoinductive material, and an antimicrobial agent. In some embodiments, the filler can include CaSO₄. In some embodiments, the osteoconductive material can include allogenic bone particles. In some embodiments, the osteoinductive material can include bone morphogenic protein (BMP, including, e.g., BMP 2 (INFUSE, Medtronic Inc., Minneapolis, Minn.) and/or BMP 7). In some embodiments, the antimicrobial agent can include metronidazole, tobramycin, gentamicin, or vancomycin.

In another aspect, this disclosure describes a method that generally includes applying an implant support composition as summarized above to a site comprising a bone defect, allowing the implant support composition to promote bone regrowth and inserting a medical device into the site.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows follow up radiographs of intervention surgery with implant support composition on failing implant from advanced peri-implantitis.

FIG. 2 shows operation radiographs and pictures of intervention surgery with implant support composition on multiple failing implants from advanced peri-implantitis.

FIG. 3 shows follow up radiographs of intervention surgery with implant support composition on multiple failing implants from advanced peri-implantitis.

### DETAILED DESCRIPTION OF ILLUSTRATIVE EMBODIMENTS

This disclosure describes an implant supporting composition. Generally, the composition can have one or more of the following properties: fast-setting, tight sealing, load-bearing, dissolvable over time, osteoconductive, and/or osteoinductive. In certain embodiments, the implant support composition can further include an antimicrobial agent.

While described below in the context of an exemplary embodiment in which implant support composition is used in a dental application, the compositions described herein can be used in other implant applications as well. Exemplary alternative implant applications include, for example, revision hip implant arthroplasty; an alternative to conventional PMMA bone cement; as a filler for bone tumor, trauma, or fusion needs (including pediatric indications); and/or as an antibiotic spacer in bone infection cases.

The compositions described herein can be used, for example, to assist rehabilitating a failing implant and/or a bony defect that in the vicinity of the failing implant (e.g., as may be due to peri-implantitis). The compositions also may be used to rescue an initially unstable dental implant that may be found, for example, in low quality bone of elderly patients or in patients with compromised healing capacity patients.

The use of dental implants can be a common method for rehabilitating missing teeth. Certain difficulties may be experienced, however, in patients with, for example, peri-implantitis and/or an implant failure in low density/volume bone.

Peri-implantitis is similar to periodontitis around natural teeth and can lead to the destruction of bone and soft tissue around an implant. Peri-implantitis is considered a common long-term complication of dental-implant therapy. It is characterized by inflammation of the mucosa and loss of supporting bone.

Lack of dense cortical bone and sparse existing hard trabecular bone as may occur, for example, in elderly patients can contribute to bone fracture during surgical drilling. Self-threading dental implants also can be unstable in this type of weak bone. When initially unstable implants are encountered, a surgeon can replace an unstable implant with a wider implant if there is enough bone available. In many cases this effort can fail, however, because the available bone may be hard but weak. In these cases, inserting a wider implant can lead to further fracture of the surrounding trabeculae and result in an unstable implant situation again. At that point, a surgeon can either remove the unstable implant or leave it in place and hope that osteointegration will occur. However, interfacial micro-motion greater than 100-150 μm can induce soft-tissue formation rather than bone formation around implants, which can result in failure of the implant.

In the context of dental application described above, exemplary embodiments of the implant compositions described herein provide an alternative option that involves using an implant support composition to stabilize an otherwise unstable implant. The composition may be time-dependent dissolvable, osteoconductive, osteoinducive, rapid-setting, and/or load-bearing. Using the composition described herein can increase initial implant stability in loose bony socket. Moreover, resorbable components of implant support composition can be gradually replaced by newly formed bone.

In some embodiments, therefore, the implant support composition may be formulated for dental applications. The phrase “formulated for dental application” refers to the collective attributes and properties desirable for the reinforcement and interventional uses as resulting from the particular combination of ingredient and their relative amounts and proportions. The desirable collective attributes and properties include, but are not limited to, high sealing capacity, optimal moldable and condensable viscosity, rapid setting time and, therefore, resistance to being washed away, biocompatibility, osteoconductivity, osteoinductivity, adequate initial load-bearing capacity, dissolvability, prophylactic antimicrobial property, and/or space maintaining capacity.

As used herein, the term “hydraulic,” when used to define the implant support composition, refers to the attribute of being hard-setting or capable of setting in water-based or aqueous environments.

Generally, the implant composition includes a filler compound that provides a moldable consistency (e.g., CaSO₄), an osteoconductive material (e.g., allogenic bone particles), an osteoinductive material (e.g., a bone morphogenic protein such as, for example, BMP 2 and/or BMP 7), and an antimicrobial agent (e.g., metronidazole, tobramycin, gentamicin, or vancomycin).

The composition can be prepared as a powder. When this powder is mixed with, for example, saline, it changes into condensable moldable consistency for easy clinical handling and sets up within approximately 10 minutes.

An implant support composition that is designed for a dental application can exhibit one or more properties that make the composition amenable for use in a dental application. Exemplary dental applications include, for example, unstable dental implants placed in low quality bone and/or peri-implantitis involving failing implants and the surrounding bony defect. The implant composition can have an initial moldable consistency when hydrated and provide tight sealing between bone and a rough surface of the implant until the implant support composition gradually dissolves over time. It can be hydraulic, hydrophilic, and condensable so that implant support composition can be delivered into a bony defect easily, then molded between alveolar bone and the dental implant to form a tight contact and seal. A tight seal at the implant-bone interface gap can prevent re-colonization of bacteria to the rough surface of dental implant, thus making re-osseointegration—new bone formation on an implant surface that has been disinfected but was once contaminated—possible in peri-implantitis treatment cases. It can have adequate viscosity and set rapidly so that the composition is not easily washed away by blood. This also permits the composition to occupy a bony defect so that soft tissue or bacteria cannot migrate back into the defect readily. These properties can also provide stability to the initially unstable implant cases. Mechanically, it can have load-bearing capacity and maintain its structure between bone and a load-bearing dental implant. Over time (e.g., over one to two months), the filler (e.g., calcium sulfate, CaSO₄) of the implant support composition can dissolve gradually and be replaced by newly formed bone. In embodiments in which the implant support composition include CaSO₄, the CaSO₄ can be resorbed by hydrolysis and can facilitate new bone formation by forming an apatite precipitation.

Further, the composition can be biocompatible, osteoconductive of traditional bone replacement materials, osteoinductive, and/or antimicrobial. An antimicrobial agent (e.g., metronidazol) and an osteoinductive component (e.g., a bone morphogenic protein such as, for example, BMP 2 and/or BMP 7) can prophylactically help to reduce the chance of infection and/or re-infection and facilitate new bone formation. Thus, much of the bony defect once occupied with implant support composition can be eventually replaced by new bone instead of soft tissue. The effects of an antimicrobial agent and an osteoinductive component can be gradual since they can be homogeneously mixed with the filler in the powder phase, but gradually released from the hydrated and hardened implant support composition into the body fluid as the filler dissolves over the time.

The implant support composition can be fast setting and have adequate initial mechanical property to permit over-building the implant support composition into bony defect to sustain its volume and shape while extra pressure is applied during suturing to achieve primary closure. Set filler of the implant support composition might promote soft tissue healing and can work as a barrier or membrane that can reduce the likelihood and/or extent to which soft tissue infiltrates into the bony defect.

In embodiments in which CaSO₄ serves as the filler component, the relatively fast dissolution of CaSO₄ and slow formation of new bone that re-occupies space created by dissolution of the filler, the volume of the composition may decrease to about 50%-70% of original volume of composition by the end of the healing period. To address this issue, the implant support composition can be over-built 20%-30% in the bony defect. Also the amount of particulate allogenic bone included in the implant support composition can be maximized (as long as moldable consistency of the implant support composition can be maintained) to maintain the volume of the implant support composition and/or serve as a scaffold where new bone can start to build. Thus, new bone formation need not be responsible to fill 100% of a bony defect. Instead, new bone can fill approximately only 50%-60% of the defect while particulate bone can occupy about 40%-50% of the defect. This can increase the chance that all of original volume of the implant support composition applied to a bony defect during the intervention surgery can be maintained after healing. The presence of an osteoinductive material (e.g., a bone morphogenic protein such as, for example, BMP 2 and/or BMP 7) in the implant support composition also can help in this regard by promoting new bone formation.

One exemplary implant support composition is shown in Table 1.

TABLE 1

| | | Amount |
|---|---|---|
| Ingredient | Purpose | (% by weight) |
| Calcium sulfate | Bioresorbable, | 71.1% |
| (CaSO₄; surgical plaster) | structural | |
| | support | |
| Particulate allogenic bone; | Scaffold, | 25.6% |
| FDBA, (0.2-1 mm | Osteoconduction | |
| particle size) | | |
| Metronidazole | Prophylactic | 1.5% |
| (antibiotic powder) | anti-infective | (pure Met. = 1%) |
| Recombinant human bone | Stimulate bone | 1.8% |
| morphogenic protein 2 | regeneration | (pure BMP = 0.1%) |
| (rhBMP2) powder with additives | | |
| Total: | | 100.0% |

W/P ratio = 0.42; Volume ratio between CaSO₄ and Bone = 1.5:1 (60%:40%)

In some embodiments, however, an alternative filler may be substituted for CaSO₄. Thus, exemplary fillers include, but are not limited to, calcium sulfate, calcium phosphate, biphasic calcium phosphate (15% HA and 85% β-TCP), tricalcium phosphate (β-TCP), calcium silicate, demineralized bone matrix (DMB), and mixtures of two or more fillers (e.g., calcium sulfate/calcium phosphate mixture, calcium sulfate/calcium phosphate/DBM mixture).

Similarly, in some embodiments, an alternative osteoconductive material may be substituted for the allogenic bone particles. Thus, exemplary osteoconductive materials include, but are not limited to, allogenic bone particles, xenograft bone particles (e.g., bovine or porcine bone particles), coral-derived granules, alloplast (e.g., synthetic hydroxyapatite, tricalcium phosphate and calcium carbonate beads, bioactive glass beads, or porous PMMA (poly methyl methacrylate)) and mixtures of two or more osteoconductive materials.

As described in more detail below, the antimicrobial agent can include metronidazole, tobramycin, gentamicin, vancomycin, tetracycline, doxycycline, minocycline, or other antibiotic, or a mixture of two or more antimicrobial agents.

In some embodiments, an alternative osteoinductive material may be substituted for bone morphogenic protein 2. Thus, exemplary osteoinductive materials include, but are not limited to, bone morphogenic protein 2 (BMP2, recombinant or native), bone morphogenic protein 7 (BMP7, recombinant or native), demineralized bone matrix (DBM), platelet-derived growth factor (PDGF, recombinant or native), enamel matrix derivative (EMD), or a combination of two or more osteoinductive materials.

The exemplary implant support composition shown in Table 1 is a combination product that has a moldable, condensable consistency due to the presence of calcium sulfate (CaSO₄). When it is applied to the bony defect and ailing dental implant, it is condensable and sets up within approximately 2-10 minutes clinically, which decreases the extent to which the composition can be washed from the site of application and increases the likelihood that the composition maintain its shape and volume during the suturing. Initially, its tight sealing property against bone and rough implant surface works as a barrier or membrane, reducing the extent of soft tissue invagination and/or the extent to which pathogenic bacteria can recolonize the site. Also, its mechanical strength can provide added stability to the load bearing dental implant without being displaced or fractured. As CaSO₄ gradually dissolves from the outer surface of implant support composition, the incorporated allogenic bone particles can become exposed and the osteoinductive material and antimicrobial agent can be released locally. This can lower the chance for reinfection and facilitate new bone formation around the osteoconductive allogenic bone particles exposed on implant support composition body. Eventually, all of the CaSO₄ can dissolve and new bone can form around the allogenic bone particles, replacing all the volume once occupied by the implant support composition at the intervention surgery.

Re-osseointegration in peri-implantitis lesions is possible but unpredictable using conventional intervention methods. This variable outcome may be due, at least in part, to the difficulty of completely eradicating bacteria from the rough surfaces of implants/infra-bony pockets. Typical particulate bone graft materials that occupy the pocket often cannot seal or prevent bacterial recolonization. Also, new bone formation around the bone-graft materials can be a slow process, which can allow bacterial biofilm to re-cover the implant surface and interfere with predictable re-osseointegration. The early sealing capacity of the implant support composition and the gradual release of the antimicrobial agent from the composition may help to reduce the number of viable bacteria on the implant surface and/or in the bony defect, thereby decreasing the likelihood and extent of reinfection. Increasing the amount of allogenic bone particles in the implant support composition while maintaining a moldable consistency can reduce the amount of new bone formation needed to replace the gradually dissolving CaSO₄. As CaSO₄ gradually dissolves away, new bone can form around the osteoconductive component (e.g., allogenic bone particles) and form a trabecular bony structure. By further incorporating an osteoinductive material (e.g., a bone morphogenic protein such as, for example, BMP 2 and/or BMP 7) into the implant support composition, the gradual release of the osteoinductive material can promote new bone formation beyond the bone growth promoted by the osteoconductive component alone. The new and allogenic bone that thereby occupies the bony defect can promote re-osseointegration in a predictable way.

Saline or an alternative solvent can be added to the implant support composition power described above to activate the components and begin the setting process. Alternative suitable solvents include, for example, distilled water or 4% potassium sulfate. In one example, a solvent/powder ratio of, for example, 0.42 (e.g., 420 mL for 1000 mg powder mixture) can be used to wet the powder phased components while maintaining a moldable consistency such as, for example, putty. After mixing, any extra water on the surface of the composition can be removed with, for example, gentle application of sterile gauze before the implant support composition is applied to the bony defect. In this state, the implant support composition is easy to work with, and can be worked into the crevices of the adjacent bone during application, yet will not flow away from the site by active bleeding during setting.

In other embodiments, the solvent:powder ratio can vary from about 0.2 to about 0.6. Factors that can influence the particular solvent:powder ratio for any given application include, for example, differences in the crystal structure of commercially available medical grade calcium sulfate products, and desired viscosity (e.g., injectable to condensable) of the resulting composition. A more viscous composition can be obtained by using a lower solvent:powder ratio. A second exemplary implant composition is shown in Table 2.

TABLE 2

| | | Amount |
|---|---|---|
| Ingredient | Purpose | (% by weight) |
| Calcium sulfate | Bioresorbable, | 69.90%-69.98% |
| (CaSO₄; surgical plaster) | structural | |
| | support | |
| Particulate allogenic | Scaffold, | 29.0% |
| bone; FDBA, (0.2-1 mm | Osteoconduction | |
| particle size) | | |
| Metronidazole | Prophylactic | 1% |
| | anti-infective | |
| Recombinant human | Stimulate bone | 0.02%-0.1% |
| bone morphogenic protein 2 | regeneration | |
| (rhBMP2) | | |
| Total: | | 100.0% |

W/P ratio = 0.55.

The injectable viscosity implant support composition shown in Table 2 with solvent/powder ratio of 0.55 can be used in, for example, revision hip arthroplasty. Revision hip arthroplasty account for close to one quarter of all arthroplasties performed. However, revisions are generally challenging due to loss of bone stock and surrounding musculature that can result from the loosening of the primary implant. Accordingly, revision hip arthroplasty can have a lower success rate (85-95% at 10 years) and/or a higher rate of complications, notably infection. Compaction of allogenic bone particles (1-3 mm in size) or use of PMMA bone cement around the revision implants can compensate for the lost bone stock, helping achieve initial implant stability, which promotes long-term implant survival. However, initial implant stability can be impaired if the allograft particles are resorbed quickly or compacted further under the cyclic loading from body movement before new bone formation and remodeling of woven bone to structurally strong lamellar bone takes place. This instability of a revision implant can allow a fibrous membrane to form at the interface and prevent osseointegration and long-term success.

Many of the challenges associated with revision surgeries can be addressed with an injectable but strong implant support composition that is a strong cement-like bone graft compound, can fill voids, and provide implant stability initially but gradually be absorbed. After absorption, the osteoconductive bony scaffold is left behind so that new bone can form around the implant and release additives to the composition such as, for example, an osteoinductive material (e.g., a bone morphogenic protein such as, for example, BMP 2 and/or BMP 7) and/or an antibiotic (e.g., tobramycin, gentamicin, and/or vancomycin). The implant support composition shown in Table 2 includes an antimicrobial agent (metronidazole) and an osteoconductive material (allogenic bone particles (ABP: 0.2-1 mm in size, 70% volume, 29% weight of implant support composition). Osteoinductive and/or antimicrobial components may not be necessary for all applications, however.

The implant support composition can provide a cement-like consistency to fill and seal the voids between cortical shell and revision orthopedic implant. The composition can set in 10 minutes to provide additional stability and compressive strength to the implant/bone construct during early phase of healing. CaSO₄ gradually dissolves from the outer bone-compound interface to the center of compound-implant interface in 4-6 weeks while leaving osteoconductive allogenic bone particles, when present in the implant support composition, as a scaffold for a new bone formation and releasing an osteoinductive material to promote new bone formation faster and/or an antibiotic to reduce the likelihood and/or extent of infection in the first six weeks. The combination of the osteoinductive material, the intrinsic osteogenic potential of CaSO₄, the amount of allogenic bone particles occupying interface space, and the fast-setting property of CaSO₄ can help achieve higher bone-implant contact percentages and bone volume fractions. Together, these properties of the implant support composition can result in improved long-term prognosis of revision surgeries compared to conventional bone cement.

A variety of conventional bone cements or replacement materials are available. Typical conventional particulated bone substitutes like bovine bone or cadaveric bone skeleton often do not make a seal around the implant surface and/or sufficiently inhibit bacterial recolonization. In initially unstable implants that are placed in weak bone cases, a conventional particulate bone material can induce further fracture of weak trabeculae when it is applied into the loose bony socket and a dental implant is inserted by rotation against it. Amorphous biologic modifiers like platelet-derived growth factor or bone morphogenic protein alone cannot occupy a peri-implantitis-induced bony defect and cannot provide mechanical support for an unstable implant. For example, orthopedic surgeons routinely remove trabecular bone before they use polymethylmethacrylate (PMMA) cement to stabilize hip implants in femur cortical shafts because the weak trabeculae can be broken by the strong PMMA and induce future instability of hip implants. PMMA can similarly be too strong to be used next to the weak trabecular bone around a dental implant. Calcium phosphate cement is known for its biocompatibility, potential resorbability, and molding capacity. It is, however, slow-setting, easily washed away, has relatively weak tensile strength, and limited resorbability, which limits its clinical application in stress-bearing areas.

The composition can include any suitable antibiotic. Various antibiotics can be effective against periodontal pathogens (e.g., anaerobic, gram negative periodontal pathogens) that induce peri-implantitis in human. Exemplary antibiotics include, for example, metronidazole, tetracycline, minocycline, and doxycycline. The pH of their solutions varies from tetracycline (pH 1.6), doxycycline (pH 2.2), minocycline (pH 3.8) to metronidazole (pH 4.5-7.0). In some embodiments, acidic to mildly neutral pH of various antibiotics may improve the solubility and functionality of the osteoinductive material (e.g., bone morphogenic protein) in the implant support composition.

### Additional Clinical Applications

The implant support composition as described in the present disclosure may be used in the context of other clinical applications.

An implant support composition can be used to stabilize initially unstable implants at first stage implant placement surgery. Initially unstable implants, after final insertion into the bony socket at the first stage surgery (implant placement surgery) can be removed and implant support composition is mixed and condensed into the loose bony socket and same implant can be replanted into implant support composition filled bony socket.

An implant support composition can be used to stabilize unstable implants at 2^{nd} stage after healing. An unstable implant that failed to achieve osseointegration after 3-6 months of healing at 2^{nd} stage surgery should be removed. Instead of placing a bone graft to the failed bony socket and waiting additional 4-6 months before another implant placement surgery is performed, immediate replantation of the failed implant with implant support composition can be performed. First, granulation tissue lining the bony socket should be thoroughly removed and the failed implant is thoroughly cleaned. The implant support composition is mixed and condensed into the bony socket and the same cleaned implant or a new implant can be replanted into the implant support composition packed bony socket.

An implant support composition can be used to treat failing implants and restore lost bone from peri-implantitis. An implant failing from peri-implantitis, which shows abnormal bony loss and signs of infection, can be treated with an implant support composition. A contaminated implant surface is de-toxificated with saline and the implant support composition is mixed and condensed into the bony defect and the exposed implant surface to create a tight seal.

An implant support composition can be used to fill and/or augment bony defects and/or maxillary sinus requiring bone grafts before or simultaneously with implant placement surgery. When a bone graft is required to augment bony defects incurred from earlier tooth extraction or to thicken the narrow alveolar ridge before the implant placement, an implant support composition can be used. For a direct sinus lift, a window of bone can be created on the lateral wall of the sinus, the sinus mucous membrane is lifted carefully without tearing the membrane, an implant with tapered healing abutment is placed immediately through the alveolar ridge and apex of the implant will penetrate into the sinus. The implant support composition is mixed and applied in a condensing manner around the implant apexes, and the soft tissue flap is sutured back. Alternatively, an indirect sinus lift may be performed through the alveolar bone using an osteotome and mallet. After initial drilling into the alveolar bone near the sinus floor, a series (from thinner to thicker) of osteotomes is used to fracture the sinus floor and tent the sinus membrane without perforation. An implant support composition is mixed and condensed into the bony socket and the implant support composition is pushed so the sinus membrane is lifted gradually by pressure without tearing, leaving the implant support composition only near the sinus floor area. The implant is self-threaded into the bony socket.

For large vertical or horizontal bony defect areas that need block bone grafts, once an autogenous block bone is positioned into the defect and fixed with screws, the deficient gaps between the recipient bony site and the block bone graft can be filled by implant support composition.

An implant support composition can be used to fill the space between an implant and an extraction socket. For immediate implant placement cases where an implant is placed immediately after a tooth extraction, a gap between the implant and extraction socket can be filled with implant support composition.

An implant support composition can be used to treat failing natural teeth and restore lost bone from advanced periodontitis. Failing natural teeth from periodontitis that show abnormal bony loss and signs of infection can be treated with an implant support composition. Granulation tissue in a defect is thoroughly cleaned and a contaminated tooth surface is thoroughly cleaned. The implant support composition is mixed and condensed into the bony defect around the failing tooth.

An implant support composition can be used for orthopedic use such as, for example, revision hip implant arthroplasty; an alternative to conventional PMMA bone cement; as a filler for bone tumor, trauma, or fusion needs (including pediatric indications); and/or as an antibiotic spacer in bone infection cases.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Implant Support Composition Preparation and Use

Implant support compositions can be prepared generally as described below, with modifications to allow for variations in certain properties (e.g., viscosity) depending on their application. One application includes intervening in the pathological condition and/or prolonging the life of an ailing implant which is losing bone surrounding the implant due to, for example, either over loading or peri-implantitis. Another application includes rescue of an initially unstable dental implant placed in low quality jawbone, e.g., in elderly or low healing capacity patients. The viscosity of implant support composition for such an application can be less viscous since the implant support composition is typically placed in a well-defined cylindrical osteotomy site prepared for implant placement and in order to reduce further chance of breaking weak trabecular bone surrounding socket wall.

The powder components of the implant support composition can be separately measured according to the weight ratio desired. All powders are mixed until homogeneous. Enough solvent (e.g., saline, distilled water, etc.) to hydrate the powder components is added to the powder components and mixed to homogeneity. In one example, the saline/powder ratio of 0.42 (420 microliters per 1 g powder mixture) is used to wet all powder phased components. After mixing, any extra water on the surface of composition mix can be removed with the gentle application of sterile gauze before the implant support composition is applied into the bony defect of peri-implantitis. In this state, the implant support composition is easy to work with, and will be condensed against the bony defect using hand instruments or sterile gauze.

### Example 2

### Clinical Case 1. Advanced Peri-Implantitis Case

A fifty-four year old healthy white female presented the advanced peri-implantitis on #20 dental implant. An original implant (4.7 mm diameter×13 mm long) was first restored in 2007 and started to show bone loss in 2009. Initial exam in 2012 by a periodontist identified 70% bone loss, 5 mm-11 mm of pocket depth, inflamed gingiva, bleeding on probing and exudates around the implant. The patient was presented with options of no treatment, experimental intervention surgery with the implant support composition, and surgical removal of implant and bone grafting and future implant placement. The patient chose to do an experimental intervention surgery with the implant support composition.

The surgical procedures followed the standard of care, removal of current crown, incision and flap elevation, granulation tissue removal, detoxification of implant surface. Once the bony defect was ready to receive the implant support composition as a regeneration material, 400 microliters of saline was mixed with 918 mg (calcium sulfate 650 mg, particulate allogenic bone 230 mg, metronidazole 10 mg, BMP2/buffers 28 mg) of powder implant support composition components. Primary closure was achieved and x-ray was taken. Medications including systemic oral antibiotics were given and post-surgery instruction was given to the patient.

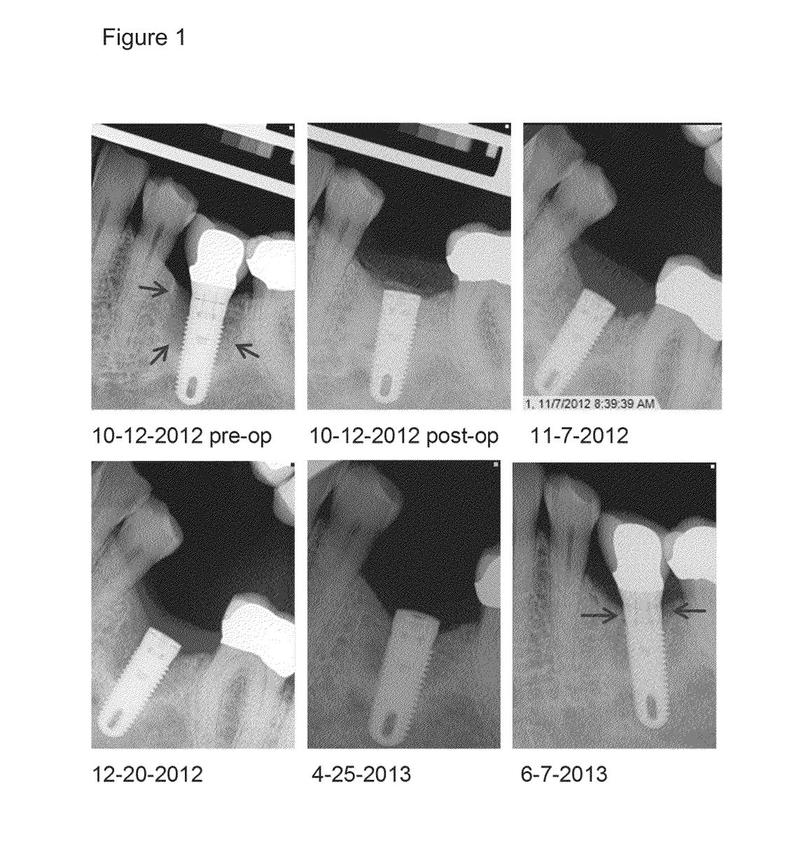

FIG. 1 shows six periapical x-ray images through eight months of follow up. In x-ray of Oct. 12, 2012 pre-op, three arrows indicate large bony defect around the #20 implant from advanced peri-implantitis. With about 70% of bone loss, current standard of care for this case would likely have been surgical removal of the failing implant and bone grafting for future implant placement. In x-ray of Jun. 7, 2013, two arrows indicate that the crest of bone (superior margin) is dense and well adapted to the implant surface, indicating success of experimental intervention surgery with the implant support composition and low chance of recurring peri-implantitis due to the tight dense bone around the implant. Patient's old crown was re-connected to the #20 implant and is functioning well. Gingiva around #20 implant is healthy and no signs of infection are present.

### Example 3

### Clinical Case 2. Advanced Peri-Implantitis on Multiple Implants

A seventy-four year old white male smoker presented multiple failing implants from peri-implantitis. Five implants (3.4 mm diameter×15 mm long) were first restored in 2004 with fixed complete denture (FCD) and patient was not seen for check up until recently. Initial exam in 2003 by a periodontist identified 100% bone loss on #22 and #26 implants, 80% bone loss on #20 implant, 30% bone loss on #24 implant. Patient had bad halitosis, deep pocket depth, severely inflamed gingiva, bleeding on probing and exudates all around the failing implants. The patient was presented with options of surgical removal of 3-5 implants, bone grafting, and future 2-5 implant placement and/or experimental intervention surgery with the implant support composition. Patient chose to do an experimental intervention surgery with the implant support composition. Two implants (#22 and #26) with no bone support were replaced with new implants supported by the implant support composition.

The surgical procedures followed the standard of care, removal of current fixed complete denture with two (#22 and #26) failed implants, incision and flap elevation, granulation tissue removal, detoxification of three implant surfaces (#20, 24, 28). Since the bony defects were too extensive, the implant support composition was prepared in three different batches. The first batch, prepared as described in Example 2, was mixed and applied around #20 implant and bottom one-third of #22 and #26 bony defects. Two new implants were connected to the sterilized fixed complete denture (FCD) at #22 and #26 locations using abutment screws. The fixed complete denture was positioned back on implants #20, 24, 28 in the patient using abutment screws while new implants #22 and #26 were positioned on top of the implant support composition pre-positioned in bony defects.

The second and third batches, prepared as described in Example 2, of the implant support composition were mixed and applied around brand new implants #22 and #26 and also around #24 and #28 implants. Multiple sutures were done around the implants and x-rays were taken. Medications including antibiotics and post-surgery instruction were given to the patient.

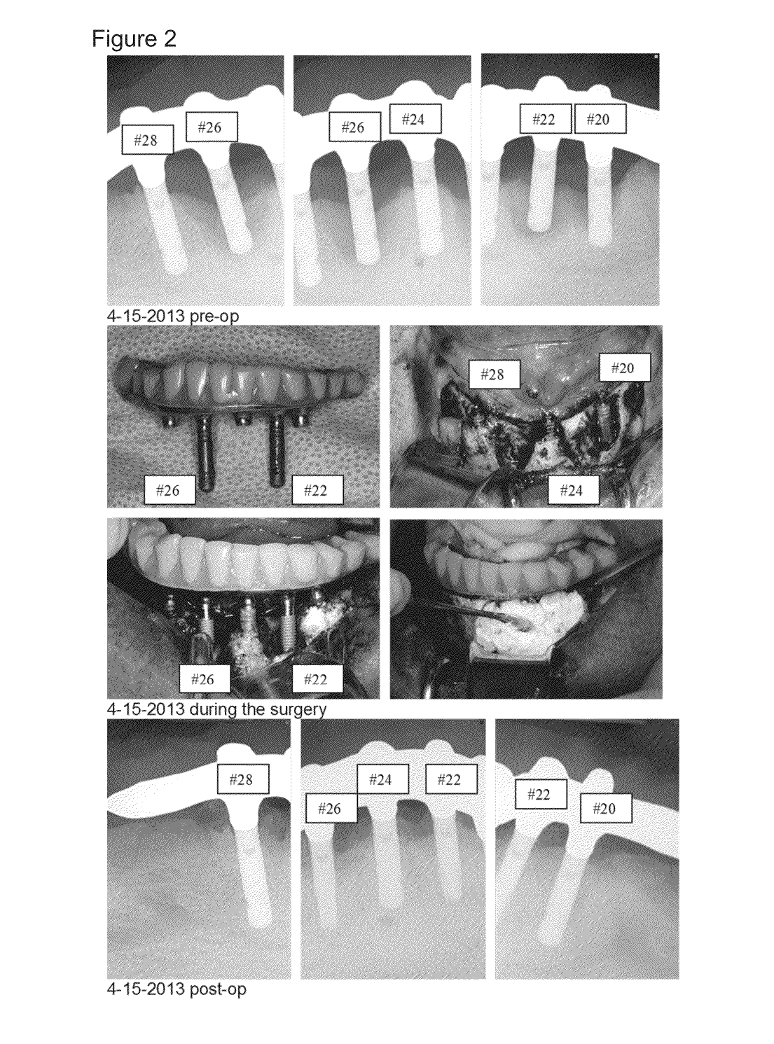

FIG. 2 shows pre-operation and post-operation periapical x-ray images and clinical pictures during the surgery. In the pre-operation x-rays, severe bone loss around #20, 22, 26 implants is evident. With that much of bone lose, current standard of care is removal of the failing implants and bone grafting for future implant placement. The two failed implants #22 and #26 came out with the fixed complete denture since there was no bone or soft tissue connections to those two implants. After fixed complete denture was removed, granulation tissue was thoroughly removed from the bony defect and exposed implant surfaces were cleaned and detoxified. Post-operation x-rays shows that implant support composition level around implants #22, 24, 26 were a bit lower than other implants, which may be a result of extra pressure applied by the surgeon due to the difficulty of suturing around fixed complete denture.

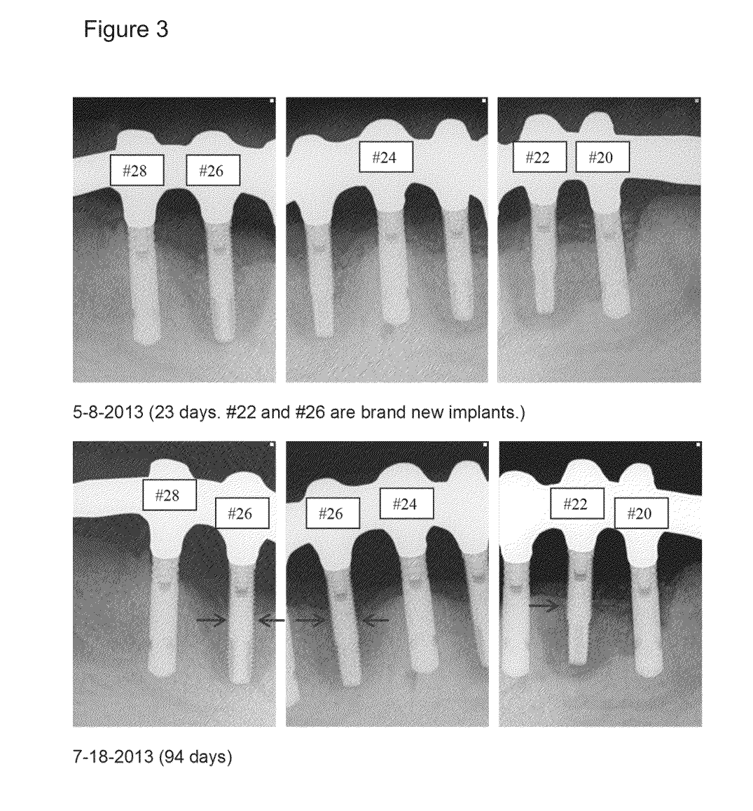

FIG. 3 shows x-ray images of follow up three months after intervention. The x-ray of May 8, 2013 shows radiodensity differences between the areas treated using the implant support composition (#20, 22, 26 implant area) and the patient's own bone area (#24 and #28 implant area). Even though patient was chewing on the fixed complete denture from the day of surgery, there is no sign of gap development between the implant support composition and two brand new implants #22 and #26. The x-ray of Jul. 18, 2013 shows two arrows per implant #22 and #26 that indicate that the crest of bone (superior margin) is becoming dense and well adapted to the implant surface, indicating new bone formation and remodeling has been initiated. Since the surgery, no signs of infection have been noticed. The patient has functioned with his current fixed complete denture (FCD) without any complications. The patient's oral hygiene has improved significantly and halitosis disappeared.

To confirm the osseointegration around implants #22 and #26, the fixed complete denture will be removed in three months and individual implant stability will be measured using an Osstell magnetic pulse device.

### Example 4

### In Vitro Studies

Functional characteristics of various embodiments of the implant support composition can be tested as follows. Test groups include (1) CaSO₄ alone, (2) CaSO₄+Particulate bone, (3) CaSO₄+Particulate bone+metronidazole, (4) CaSO₄+Particulate bone+BMP, (5) CaSO₄+Particulate bone+metronidazole+BMP.

### Characterization of Implant Support Composition

(1) Hydrated and set implant support composition (ISC) disc of 10 mm in diameter by 2 mm thick and (2) simulated human mandible-ISC-implant specimen cross-sectioned by Isomet micro-saw is examined by scanning electron microscope (SEM) for morphology, surface characteristics, and interface characteristics of the implant support compositions; energy dispersive x-ray spectroscopy (EDS) for quantitative element analysis and chemical characterization; x-ray diffraction (XRD) for crystal structure and compositions of implant support composition; and Fourier transform infrared spectroscopy (FT-IR) for functional group identification.

### Solubility Test

An implant support composition disc of 10 mm in diameter by 2 mm thick is immersed in phosphate buffered saline (PBS) and subjected to conventional assays for measuring (1) the implant support composition disc percentage weight change (solubility in water) over the time, (2) pH change in extraction medium of the implant support composition over the time, (3) BMP 2 release detection in extraction medium using ELISA kit, (4) metronidazole release in extraction medium using either ELISA kit or UV-vis spectroscopy.

### Cytotoxicity, Antibacterial Activity, Alkaline Phosphatase (ALP) Activity

The implant support composition extraction medium is used to test (1) human fetal osteoblast (hfOB)'s viability, (2) antibacterial activity on peri-implantitis pathogens, and (3) released BMP 2's effect on early differentiation of hfOB by measuring ALP activities (Alkaline phosphatase kit).

### Initial and Final Setting Time and Compression Strength

Samples of 5 mm in diameter by 10 mm tall are tested for their compression strength. A Gilmore needle (1 mm in diameter) apparatus of 300 g is used on 5 mm in diameter by 5 mm thick implant support composition discs to measure initial and final setting times.

### Setting Time

Implant bone compositions were prepared as described in Example 1, each having the composition shown in Table 3.

Results are shown in Table 3

TABLE 3

| | | Average | Average | |
|---|---|---|---|---|
| | | initial | final | |
| | w/p | setting | setting | Compression |
| | ratio | time | time | strength |
| | (mL/g) | (min:sec) | (min:sec) | (MPa) |
| CaSO₄ (100 wt %) | 0.6 | 1:47 | 5:12 | 9.39 |
| CaSO₄ (73.5 wt %) + | 0.42 | 9:47 | 30:12 | 9.01 |
| ABP (26.5 wt %) | | | | |
| CaSO₄ (71.1 wt %) + | 0.42 | 8:57 | 32:01 | 3.73 |
| ABP (25.6 wt %) + | | | | |
| BMP (1.8 wt %, pure | | | | |
| 0.1%) + metronidazole | | | | |
| (1.5 wt %, pure 1%) | | | | |

### Example 5

### Clinical Application 1: To Treat a Failing Implant from Peri-Implantitis

A failing implant with a continuous bone loss around it due to overloading and/or peri-implantitis is treated using an implant support composition is prepared, generally as described in Example 1 at the time of the procedure.

Following standard periodontal surgical protocol, full thickness flap of soft tissue is opened to uncover implant threads and expose surrounding bone. Implant-supported restoration is removed and cover-screw is connected to the implant top securely. Granulation tissue surrounding the implant and occupying infra-bony socket of surrounding bone is thoroughly removed by curette. The surface of exposed threads of dental implant is detoxified (e.g., with saline, citric acid, chlorhexidine, ethylene diamine tetraacetic acid (EDTA), hydrogen peroxide, tetracycline, etc.) as the implant support composition powder composition ingredients are being mixed.

For a 0.5 cc bony defect, the implant support compound includes calcium sulfate (medical grade) 650 mg, allogenic particulate bone 230 mg, metronidazole 10 mg, BMP2/buffers 30 mg are mixed homogeneously in a sterile porcelain cup. 400 microliters of sterile saline is added to the cup and the implant support composition is mixed thoroughly.

Meanwhile, a soft tissue flap is reflected and the detoxified surface of an ailing implant and surrounding bone socket are exposed. The implant support composition is packed (using a #7 spatula or large amalgam plugger and sterile gauze) into the infra-bony pockets thoroughly so that there is no empty space left in sulcus.

After cleaning any extra implant support composition remnant from the bone, the implant-supported crown restoration and abutment are reconnected to the implant. The soft tissue flap is sutured back around the dental implant and the occlusion of the failing implant is carefully adjusted so that no further occlusal trauma can be applied. Post-surgical X-ray is performed and prescriptions for systemic antibiotics and 0.2% chlorhexidine digluconate rinse are given to the patient.

### Example 6

### Clinical Application 2: To Rescue an Initially Unstable Dental Implant

A bony socket is prepared following standard dental implant surgical protocol using sequential drills, a self-threading dental implant is inserted using slow engine of 15-30 rpm. If the implant is unstable at the bony socket and the prognosis of success for the implant is low even after healing, an implant support composition is used to rescue an initially mobile, possibly failing implant.

The unstable implant is removed from the bony socket and rinsed with saline to remove surface blood, and then dried with an air syringe. A tapered cover screw is connected to the implant top securely with a screw driver. Using a sterile gauge, the bony socket is compressed to control bleeding while the implant support composition is being mixed accordingly.

Calcium sulfate (medical grade) 650 mg, allogenic particulate bone 230 mg, metronidazole 10 mg, BMP 2/buffers 20 mg-30 mg are mixed homogeneously in sterile porcelain cup. 400 microliters sterile saline is added to the cup and the implant support composition is mixed thoroughly.

The gauze compaction is removed and the bony socket is exposed. Molded implant support composition mixture is placed into the bony socket and condensed further into the socket using an amalgam plugger and a gauze on top of the implant support composition.

The retrieved dental implant is rotated back into the bony socket where the implant support composition has been applied until the tapered cover screw touches the brim of cortical bone and the implant is stabilized. After thoroughly cleaning remaining excess implant support composition remnant from around the implant and surrounding bone, the soft tissue flap is sutured to cover the implant completely. A post-surgical x-ray is performed and instructions dispensed to the recipient/patient.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

Terms such as “about,” “generally,” “substantially” and the like are to be construed as modifying a term or value such that it is not an absolute, but does not read on the prior art. Such terms will be defined by the circumstances and the terms that they modify are understood by those of skill in the art. This includes at the very least the degree of expected experimental error, technique error, and instrument error for a given technique used to measure a value.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. An implant support composition comprising:
a filler; and
an osteoconductive material.

2. The implant support composition of claim 1 wherein the filler comprises CaSO₄.

3. The implant support composition of claim 1 wherein the osteoconductive material comprises allogenic bone particles.

4. The implant support composition of claim 1 further comprising an osteoinductive material.

5. The implant support composition of claim 4 wherein the osteoinductive material comprises bone morphogenic protein.

6. The implant support composition of claim 4 further comprising an antimicrobial agent.

7. The implant support composition of claim 6 wherein the antimicrobial agent comprises metronidazole, tobramycin, gentamicin, or vancomycin.

8. The implant support composition of claim 1 further comprising an antimicrobial agent.

9. The implant support composition of claim 8 wherein the antimicrobial agent comprises metronidazole, tobramycin, gentamicin, or vancomycin.

10. The implant support composition of claim 1 wherein the implant support composition is capable of setting in no more than 10 minutes.

11. The implant support composition of claim 10 wherein the implant support composition is capable of setting in no more than two minutes.

12. A method comprising:
applying an implant support composition to a site comprising a bone defect, wherein the implant support composition comprises:
a filler; and
an osteoconductive material;
allowing the implant support composition to promote bone regrowth; and
inserting a medical device into the site.

13. The method of claim 12 wherein the implant support composition further comprises an osteoinductive material.

14. The method of claim 13 wherein the composition further comprises an antimicrobial agent.

15. The method of claim 12 wherein the composition further comprises an antimicrobial agent.

16. The method of claim 12 further comprising:
allowing the composition to set for no more than 10 minutes after inserting the medical device.

17. The method of claim 12 further comprising:
allowing the composition to set for no more than two minutes after inserting the medical device.
